(19) United States
(12) Patent Application Publication
(10) Pub. No.: US 2020/0016235 A1
(43) Pub. Date: Jan. 16, 2020

(54) HIGHLY CONCENTRATED FORMULATIONS AND METHODS FOR PRODUCING LARGE BATCH PHARMACEUTICAL FORMULATIONS

(71) Applicant: Parenteral Technologies, LLC, Siesta Key, FL (US)

(72) Inventor: Michael A. Creaturo, Siesta Key, FL (US)

(21) Appl. No.: 16/509,025

(22) Filed: Jul. 11, 2019

Related U.S. Application Data
(60) Provisional application No. 62/696,598, filed on Jul. 11, 2018.
(60) Provisional application No. 62/798,604, filed on Jan. 30, 2019.
(60) Provisional application No. 62/840,572, filed on Apr. 30, 2019.

(51) Int. Cl. A61K 38/12; A61K 31/7036; A61K 9/08; A61K 9/00; A61K 31/573; A61K 31/7048

(52) U.S. Cl. CPC A61K 38/12; A61K 31/7036; A61K 31/7048; A61K 9/0048; A61K 31/573; A61K 9/08

(57) ABSTRACT

A highly concentrated formulation and method for producing a batch of a pharmaceutical formulation utilizing the same, the highly concentrated formulation containing at least one active pharmaceutical ingredient (API) admixed with a first inactive excipient and having a first API potency to produce the a pharmaceutical formulation having a second API potency less than the first and in compliance with a predetermined regulatory pharmacopeia or formulary monograph for the produced pharmaceutical formulation, the highly concentrated formulation contained in a container labeled with the at least one API, the second inactive excipient paired for use with the highly concentrated formulation, the pharmaceutical formulation to be produced, and the amount of the pharmaceutical formulation to be produced.

### CROSS-REFERENCE AND INCORPORATION BY REFERENCE

This application claims the benefit of priority of U.S. Provisional Application No. 62/696,598 filed Jul. 11, 2018, U.S. Provisional Application No. 62/798,604 filed Jan. 30, 2019, and U.S. Provisional Application No. 62/840,572 filed Apr. 30, 2019, the contents of each of which are incorporated by reference herein.

### BACKGROUND

Over the counter (OTC) and prescription (Rx) products are required to meet strict monograph regulatory requirements specifying active pharmaceutical ingredient (API) potency and excipient formulation. To meet these requirements, it is necessary to perform specific compounding and processing steps for active pharmaceutical ingredients and excipients when preparing formulations.

For example, conventional processes for producing an OTC or Rx pharmaceutical topical or ophthalmic product require that each API be procured, received, tested, quality control evaluated, and released. Each API active microbial or chemical purity must also be accurately tested and identified. Each API purity must also be converted for use in a ratio calculation for the introduction into an excipient to achieve a predetermined potency ratio required for a specific formulation, typically from a regulatory monograph. The calculated ratio of each API, typically in a powdered or crystalline form, is introduced into an excipient during a blending process and likely also a heating process for homogenization. The formulation including the blended API(s) is then admixed with other ingredients and excipients, typically in a large volume vessel tank for final filling and packaging. Remaining or excess API may be re-sealed and stored for later use.

Conventional manufacturing processes for producing a batch formulation are further exacerbated for each additional API. Thus, introducing a second API to a formulation typically doubles the hands-on procedures, while a third API triples those same procedures, etc. In addition, the process of receiving multiple APIs, quality control, regulatory procedural requirements, calculations, and the subsequent potency of each API to be admixed requires skill in compounding and other trade craft knowledge. More specifically, accurately and effectively producing a correct potency mix of multiple APIs requires multiple calculations, testing and exact mixing. Furthermore, to effectively activate a vessel containing a predetermined volume of excipient to meet a specific regulatory formulation monograph requires that the correct potency of each API and respective volume ratio be calculated and correlated specifically to the volume of other excipients within the vessel. These procedures require advanced compounding skill and trade craft knowledge.

Typically, most APIs are produced in a final powder or crystalline form. As such, introducing APIs into an excipient in such a form to obtain a uniform distribution and dissolution of the APIs further complicates the manufacturing process. Solubility and particle size are important characteristics that must be controlled to assure potency uniformity in topical drug products such as emulsions, creams, and ointments. Crystalline form is also important where the active ingredient is dispersed as a solid phase in either the oil or water phase of an emulsion, cream, or ointment. As such, the final particle size of the APIs is critical and must be controlled. Particle size may also affect the activity of the drug substance, as the smaller a particle size the greater the surface area. Particle size may also affect the degree to which the product may be physically irritating when applied, generally with smaller particles being less irritating than larger particles.

It is therefore important that API solubility in a carrier be known, quantified, and validated during manufacturing where the API is added to a large quantity of excipient. Poor mixing or lack of oversight and investigation into particle size distribution and solubility of APIs introduced into a vessel must be evaluated to avoid “dead spots” where quantities of the formula are stationary and not subject to mixing, as potency uniformity is based largely upon adequate distribution of the component throughout the mix.

Production controls must therefore be implemented that account for the solubility characteristics of the APIs and their introduction into the excipient within the vessel, as inadequate controls can adversely affect final product potency, efficacy, and safety. Combining two or more APIs as a powder, solution, gel, or semi-solid to create a stable long-term formulation in most cases is inherently difficult, especially in the case of antibiotics of different classes. Preserving and preventing the degradation of the microbial assay, among others physical factors, such as pH, loss on drying, specific gravity and total viable aerobic microbial count of each API, combined within the excipient, requires formulation trials and studies. Studies must examine many facets including stability under different temperatures, moisture, pH, among other environmental conditions, only then to be further examined for potential chemical reactions, adverse interactions and/or degradation of one or more of the APIs within the excipient.

Accordingly, what is needed are new, more efficient and cost-saving processes for producing batch pharmaceutical formulations.

### BRIEF SUMMARY

To achieve the foregoing and other advantages, in one aspect, the inventive concepts disclosed herein are directed to a packaged formulation for producing a large batch pharmaceutical formulation, the packaged formulation including a container, a highly concentrated formulation contained in the container, the highly concentrated formulation comprising at least one active pharmaceutical ingredient (API) admixed with a first inactive excipient, the highly concentrated formulation having a first API potency to produce a pharmaceutical formulation having a second API potency less than the first API potency and in compliance with a predetermined regulatory pharmacopeia or formulary monograph for the produced pharmaceutical formulation when the highly-concentrated formulation is admixed with a second inactive excipient, and indicia on the container indicating the at least one API, the second inactive excipient paired for use with the highly concentrated formulation, the pharmaceutical formulation to be produced, the second API potency, and an amount of the second inactive excipient or the pharmaceutical formulation to be produced when the highly concentrated formulation is admixed with the second inactive excipient.

In some embodiments, the first API potency is at least 30 times greater than the second API potency, preferably at least 50 times greater than the second API potency, and more preferably at least 100 times greater than the second API potency.

In some embodiments, a volume of the second inactive excipient is at least 30 times greater than a volume of the first inactive excipient, preferably at least 50 times greater than a volume of the first inactive excipient, and more preferably at least 100 times greater than a volume of the first inactive excipient.

In some embodiments, the first inactive excipient and the second inactive excipient are the same and include one or more excipients, and at least one of the first inactive excipient and the second inactive excipient is a liquid, a gel, a semi-solid, or a solid at room temperature.

In some embodiments, the container includes a discharge port through which the highly concentrated formulation is discharged from the container, and optionally includes at least one of a spout and a cap.

In some embodiments, the pharmaceutical formulation is a finished (e.g., ready to use) topical or ophthalmic formulation product.

In some embodiments, the container is marked with a barcode, for example a quick response (QR) code, containing information about the highly concentrated formulation, the pharmaceutical formulation to be produced, the paired second inactive excipient, etc.

In some embodiments, the highly concentrated formulation includes 1, 2, 3, 4 . . . n number of APIs and 1, 2, 3, 4 . . . n number of inactive excipients.

In another aspect, the inventive concepts disclosed herein are directed to a method for producing a pharmaceutical formulation. The method includes providing a packaged formulation including a container, a highly concentrated formulation contained in the container, the highly concentrated formulation comprising at least one active pharmaceutical ingredient (API) admixed with a first excipient, the highly-concentrated formulation having a first API potency to produce a pharmaceutical formulation having a second API potency less than the first API potency and without correlation, calculation or compounding yielding a pharmaceutical formulation in compliance with a predetermined regulatory pharmacopeia or formulary monograph for the produced pharmaceutical formulation when the highly-concentrated formulation is admixed with a second inactive excipient, indicia on the container indicating the at least one API, the second inactive excipient paired for use with the highly concentrated formulation, the pharmaceutical formulation to be produced, the second API potency, and an amount of the second inactive excipient or the pharmaceutical formulation to be produced when the highly concentrated formulation is admixed with the second inactive excipient, and admixing the highly concentrated formulation with the second excipient contained in a vessel, wherein an amount of the second excipient is greater than an amount of the first excipient.

In some embodiments, the amount (e.g., mass or volume) of the second inactive excipient is greater than the amount of the first inactive excipient by a factor of at least 30, preferably at least 50, more preferably at least 100.

In some embodiments, the first API potency is at least 30 times greater than the second API potency, preferably at least 50 times greater, more preferably at least 100 times greater.

Embodiments of the inventive concepts can include one or more or any combination of the above features.

### BRIEF DESCRIPTION OF THE DRAWINGS

Implementations of the inventive concepts disclosed herein may be better understood when consideration is given to the following detailed description thereof. Such description makes reference to the included drawings. Like reference numerals in the drawings may represent and refer to the same or similar element, feature, or function. In the drawings:

FIG. 1 depicts an ABL or other composite foil pouch for containing a highly concentrated formulation according to the present disclosure;

FIG. 2 depicts an HDPE or other composite plastic jerry container for containing a highly concentrated formulation according to the present disclosure;

FIG. 3 depicts an aluminum or other metal composite tray for containing a highly concentrated formulation according to the present disclosure;

FIG. 4 depicts a cardboard or other fiber or paper container with a bag insert for containing a highly concentrated formulation according to the present disclosure; and

FIG. 5 is a flowchart illustrating a method for producing a pharmaceutical formulation utilizing a highly concentrated formulation according to the present disclosure.

### DETAILED DESCRIPTION

The inventive concepts are described hereinafter with reference to the accompanying drawings in which exemplary embodiments are shown. However, the inventive concepts may be embodied in many different forms and should not be construed as limited to the representative embodiments set forth herein.

Disclosed herein are products, methods and processes that eliminate, at certain stages of the production process, the need for repetitive hands-on procedures by providing an enhanced pre-mixed active pharmaceutical ingredient (API) composition, also referred to hereinafter as a “highly concentrated formulation.” The highly concentrated formulation advantageously reduces and/or eliminates multiple repetitive procedures associated with each API including procurement, quality control, regulatory control, validation, solubility trials and studies, calculation of API purity to volume, API mixing, compounding procedures, and the processing and calculation of API individual potency ratio to the volume of excipient(s) within a vessel or other holding container or to be transferred to another vessel to achieve a specific monograph potency parameter for a final pharmaceutical formulation (i.e., finished product ready to be packaged and used), such as a topical or ophthalmic formulation.

The highly concentrated formulation may contain 1, 2, 3, 4 . . . n number of APIs paired to a specific type and volume of excipient(s) within a vessel. The introduction of the highly concentrated formulation specifically activates and achieves the predetermined potency ratio of the API(s), and thus subsequent potency levels within the predetermined volume of inactive or inert excipient(s) contained in the vessel, without the need for conventional procedures such as compounding, calculating and analytical testing of API(s) to obtain correct potency, among others. Further, upon introduction into a vessel containing a paired excipient volume, the highly concentrated formulation accurately activates the paired excipient to a predetermined potency of the API(s) to meet a specific OTC or Rx monograph parameter for a particular pharmaceutical formulation.

Disclosed herein are methods for homogenizing a predetermined volume of a highly concentrated formulation including a predetermined volume of at least one excipient, optionally a viscosity agent or preservative, and a predetermined volume of at least one API to provide the highly concentrated formulation having a predetermined API potency concentration level, that when introduced to a predetermined paired volume of an excipient(s) produces a product meeting a set OTC or Rx regulatory formulation drug monograph parameter for API potency level within the second composition.

The excipient and/or viscosity agent of the highly concentrated formulation may be an ingredient listed within the produced pharmaceutical formulation's original regulatory monograph, thus harmonizing and further limiting additional regulatory requirement.

The highly concentrated formulation is labeled and to be added to a predetermined volume of a paired excipient, that when added and homogenized, produces an overall predetermined potency of the API(s) to meet an OTC or Rx drug monograph for a particular formulation, such as a topical or ophthalmic formulation as indicted and labeled on the pharmaceutical formulation packaging or package insert.

The highly concentrated formulation may be in the form of a liquid, a powder, a gel, a slurry, a semi-solid, or a solid at room temperature, and may optionally contain one or more preservatives or dispersal agents.

Preferable APIs used in the preparation of the highly concentrated formulation are ultra-high purity and may be produced solely for use in the highly concentrated formulation, thus reducing the volume of APIs necessary within the highly concentrated formulation and the overall amount of the highly concentrated formulation required to achieve the same API potency levels within the second excipient as compared to conventional industry practice. Such ultra-high purity APIs may have 5%, 10% or 20% greater purity than current commercially available APIs and used in typical production that are in compliance with USP, BP or other regulatory monograph for those APIs.

The highly concentrated formulation may be formulated as a premixed liquid having a stable predetermined volume and API potency concentration level to facilitate efficient blending, dissolution, and distribution of the contained APIs within the paired excipient. The highly concentrated formulation provided as a premixed liquid composition obviates the need for calculating, compounding, and admixing individual API powders to the paired excipient.

The highly concentrated formulation may have a low melting point such that, at or around ambient temperature, the highly concentrated formulation has the physical characteristics of a solution, semi-solid gel, oil, etc., thereby protecting and/or enhancing stability of the at least one API contained therein. The excipient and/or viscosity agent may be solid or semi-solid at ambient temperature and may be heated to become free flowing to facilitate agitation and mixing prior to the addition of the at least one API. One or more of the excipient and/or viscosity agent of the highly concentrated formulation may stabilize, among other attributes, the API purity, pH, specific rotation, assay, and dissolution of the highly concentrated formulation. One or more of the excipient and/or preservative of the highly concentrated formulation may be a salt, solvent, paraben, sorbate, sulphite, acid, alcohol, chloride, urea, cresol, phenol or salicylate compound, as well as mixtures and derivatives thereof, among others.

In some embodiments, the excipient of the highly concentrated formulation may include pharmaceutically-acceptable preservatives such as sodium chloride, sodium acetate, sodium phosphate, potassium chloride, thimerosal, trimethoprim, boric acid, EDTA, propylene glycol, glycerol, mannitol, sorbitol, dextrose, lactose polyethylene glycol, lactic acid, lactate salt benzylparaben, butylparaben, ethylparaben, isobutylparaben, isopropylparaben, methylparaben, propylparaben, or any of their salt, sodium methylparaben and sodium propylparaben, ascorbic acid, ascorbyl palmitate, benzoic acid, butylated hydroxyanisole, butylated hydroxytoluene, calcium ascorbate, calcium propionate, calcium sorbate, caprylic acid, dilauryl thiodipropionate, erythorbic acid, gum guaiac, glutathione, potassium bisulfite, potassium metabisulfite, potassium sorbate, propionic acid, propyl gallate, sodium ascorbate, sodium benzoate, sodium bisulfite, sodium metabisulfite, sodium propionate, sodium sorbate, sodium sulfite, sorbic acid, stannous chloride, sulfur dioxide, thiodipropionic acid, and/or tocopherols, or mixtures thereof and/or derivatives thereof.

In some embodiments, the excipient of the highly concentrated formulation may be serve as a buffer providing for a stability of the pH of the highly concentrated formulation as well as potency, among other attributes.

In some embodiments, the excipient of the highly concentrated formulation may obtain stability of the highly concentrated formulation in a homogeneous solution or gel, among others, such as when formulated in an acetate or citrate buffer and derivatives thereof, among others.

In some embodiments, the excipient of the highly concentrated formulation may resist or prevent microbial contamination.

In some embodiments, the excipient of the highly concentrated formulation may include an amino-acid such as glycine, alanine, lysine, proline, histidine and tryptophan, among others.

In some embodiments, the excipient of the highly concentrated formulation may be a natural oil or synthetically derived oil such as cotton seed oil, olive oil or mineral oil, as well as mixtures thereof, among others.

In some embodiments, the excipient of the highly concentrated formulation may be a hydrocarbon derivative such as paraffin wax, microcrystalline wax, white mineral oil, petrolatum or other petroleum derivative.

In some embodiments, the viscosity unit measurement of the highly concentrated formulation may be formulated to be not less than 150,000, 250,000 or 300,000 Centipoise (cP).

In some embodiments, the viscosity unit measurement of the highly concentrated formulation may be formulated to be greater than 3 Centipoise (cP).

In some embodiments, the viscosity unit measurement of the highly concentrated formulation may be introduced to create a produced pharmaceutical formulation with the vessel excipient of not less than 200,000, 250,000 or 300,000 Centipoise (cP).

In some embodiments; the amount of packaged highly concentrated formulation in in the range from 1 kg to 100 kg, more preferably 5 kg to 30 kg, even more preferably 10 kg to 20 kg, and most preferably around 20 kg.

In some embodiments, the excipient of the highly concentrated formulation may be a naturally derived ester such as cetyl palmitate derived from cetyl alcohol and palmitic acid or other a naturally occurring fatty acid derived from plants or animals.

In some embodiments, the excipient of the highly concentrated formulation may be a synthetic or semi-synthetic derived product such as sodium pyruvate, tocopheryl acetate, etc.

In some embodiments, the excipient of the highly concentrated formulation may be a natural or synthetic wax emulsion derived product such as beeswax, carnauba wax, paraffin wax, etc.

In some embodiments, the excipient of the highly concentrated formulation may be a cetomacrogol or other emulsifying wax, liquid paraffin, methylparaben, propylparaben, petrolatum or petroleum products, etc.

In some embodiments, the highly concentrated formulation may have a melting point around 37° C. to 38° C., or a melting point greater than about 74° C.

In some embodiments, the highly concentrated formulation may be free-flowing above a temperature of about 55° C., and may be a solid at a temperature below about 32° C.

In some embodiments, the highly concentrated formulation may have a melting point between about 33° C. and 39° C. and may be a solid at a temperature below about 33° C.

In some embodiments, the API of the highly concentrated formulation may be an anti-infective ingredient, anti-fungal ingredient, steroid, hormone, corticosteroid, etc.

In some embodiments, the API of the highly concentrated formulation may be micronized to enhance distribution and solubility properties within the vessel excipient.

In some embodiments, the API of the highly concentrated formulation may have a predetermined mesh profile to assist and enhance the dissolution and distribution of the API within the vessel excipient.

In some embodiments, the highly concentrated formulation may contain one or more dissolution agents, among other compounds, to assist and enhance API dissolution and distribution within the vessel excipient.

In some embodiments, the highly concentrated formulation may contain agents or compositions to assist and enhance the encapsulation of the API, and/or the dissolution and distribution of the API, within the vessel excipient.

In some embodiments, the API may include at least one of Neomycin Sulfate, Polymyxin B Sulfate, and Bacitracin or Bacitracin Zinc.

In some embodiments, the API may include one or more of Neomycin, Gramicidin, Nystatin, and Triamcinolone.

In some embodiments, the API may include one or more of antibiotic, antifungal, anesthetic, hormonal, steroidal, among other therapeutic APIs and/or active therapeutic compounds.

### Examples of Highly Concentrated Formulations

A first exemplary highly concentrated formulation includes Neomycin Sulfate, Polymyxin B Sulfate and Bacitracin, in predetermined amounts and/or ratios to achieve a potency concentration in a predetermined volume of excipient in a vessel to achieve a final formulation range of about 70% to 130% of label indicating a potency of; Neomycin Sulfate, 3.5 mg; Polymyxin B Sulfate, 5,000 units; Bacitracin or Bacitracin Zinc, 400 units per gram.

Another exemplary highly concentrated formulation includes Neomycin Sulfate, Polymyxin B Sulfate and Bacitracin, in predetermined amounts and/or ratios to achieve a potency concentration in a predetermined volume of excipient in a vessel to achieve a potency levels, among others, in a final formulation of; Neomycin Sulfate, 3,500 mg; Polymyxin B Sulfate, 5,000,000 units; Bacitracin or Bacitracin Zinc, 400,000 units per kilogram of excipient volume.

Yet another exemplary highly concentrated formulation includes Neomycin Sulfate, Polymyxin B Sulfate and Bacitracin, in predetermined amounts and/or ratios to achieve a potency concentration in a predetermined volume of excipient in a vessel to achieve a potency, among others, in the final formulation of; Neomycin Sulfate, 3.00-5.00 mg per gram; Polymyxin B Sulfate, 8,000-14,000 units per gram; Bacitracin or Bacitracin Zinc, 400-700 units per gram.

Yet another exemplary highly concentrated formulation includes Neomycin Sulfate, Polymyxin B Sulfate and Bacitracin, in predetermined amounts and/or ratios to achieve a potency concentration in a predetermined volume of excipient in a vessel of formulation percentages less than 1.0% Neomycin Sulfate; Polymyxin B Sulfate; Bacitracin or Bacitracin Zinc of the total excipient volume.

Yet another exemplary highly concentrated formulation includes Neomycin Sulfate, Polymyxin B Sulfate, Bacitracin and Pramoxine, in predetermined amounts and/or ratios to achieve a potency concentration in a predetermined volume of excipient in a vessel to achieve a final formulation range of about 70% to 130% of a label indicating; a total of approximately 1.0% of Pramoxine within the formulation, and Neomycin Sulfate, 3.5 mg; Polymyxin B Sulfate, 5,000 units; Bacitracin or Bacitracin Zinc, 400 units per gram.

Yet another exemplary highly concentrated formulation includes Neomycin Sulfate, Polymyxin B Sulfate and Bacitracin, in predetermined amounts and/or ratios to achieve a concentration level in a predetermined volume of excipient to achieve a final formulation having less than 1.0% of Neomycin Sulfate; Polymyxin B Sulfate; Bacitracin or Bacitracin Zinc with an excipient volume percentage greater than 90%.

Yet another exemplary highly concentrated formulation includes Neomycin Sulfate, Polymyxin B Sulfate and Bacitracin, in predetermined amounts and/or ratios to achieve a potency concentration in a predetermined volume of excipient in a vessel to achieve a final formulation having less than 1.0% Neomycin Sulfate, less than 1.0% Bacitracin or Bacitracin Zinc, and less than 0.5% Polymyxin B Sulfate of the total excipient volume.

Yet another exemplary highly concentrated formulation includes Neomycin Sulfate, Polymyxin B Sulfate and Bacitracin, in predetermined amounts and/or ratios to achieve a potency concentration in a predetermined volume of excipient in a vessel to achieve a final formulation having less than 0.8% Neomycin Sulfate, less than 1.0% Bacitracin or Bacitracin Zinc, and less than 0.3% Polymyxin B Sulfate, and an excipient volume percentage greater than 94%.

Yet another exemplary highly concentrated formulation includes Neomycin Sulfate, Polymyxin B Sulfate and Bacitracin are provided in respective volumes to achieve a 100% potency activity within the complete contents of a predetermined volume of excipient.

Yet another exemplary highly concentrated formulation includes Neomycin Sulfate, Polymyxin B Sulfate and Bacitracin, provided in predetermined amounts and/or ratios to achieve at a 100% activity basis in a predetermined volume of excipient with percentages of about 0.3 to 0.7% Neomycin Sulfate, about 0.05 to 0.5% Polymyxin B Sulfate, and 0.5 to 1.0% Bacitracin.

Yet another exemplary highly concentrated formulation includes Neomycin Sulfate, Polymyxin B Sulfate and Bacitracin in a predetermined potency concentration level within the premix to achieve the activation of the complete contents of a specific volume of excipients in a vessel to achieve a final formulation percentage adjusted to a 100% activity basis and an excipient volume percentage of about 93% to 99%.

Weight percentages of APIs in the highly concentrated formulation may be adjusted in terms of at least one of ratio, potency and volume, and subsequently labeled as such and for exclusive use with a vessel containing a predetermined volume of second paired excipient, for example, from 10 liters to 500,000 liters, or adjusted to a volume, such as 10 to 10,000 kgs of excipient volume within a vessel.

### Packaging

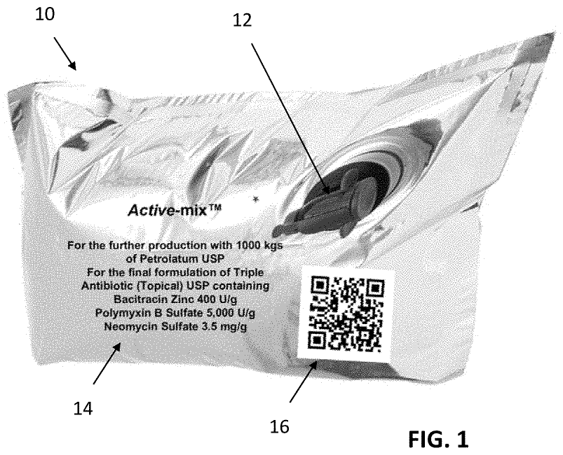

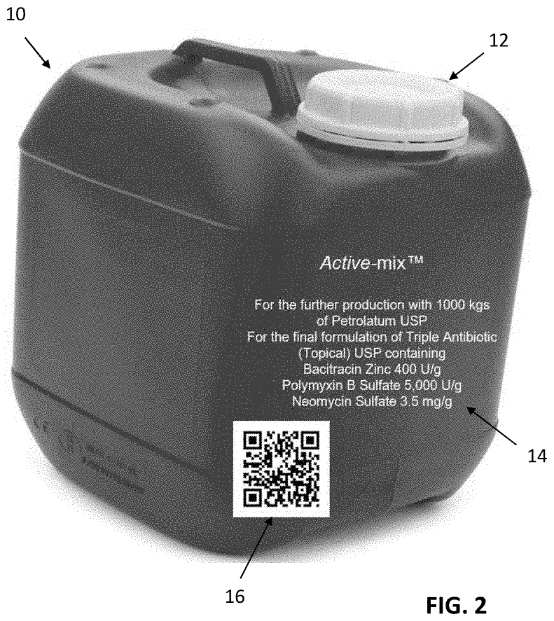

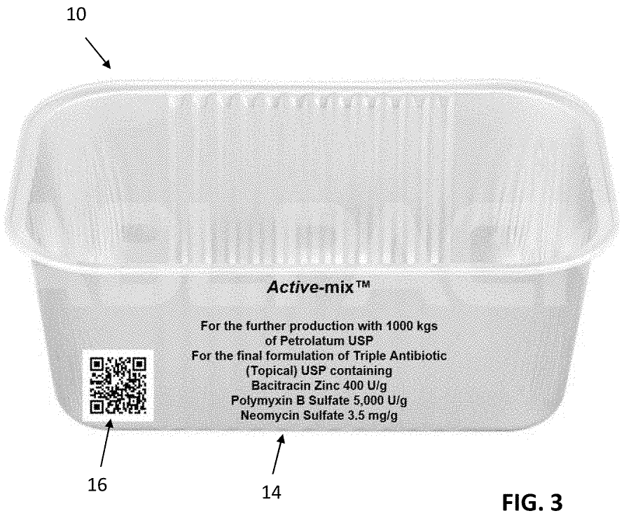

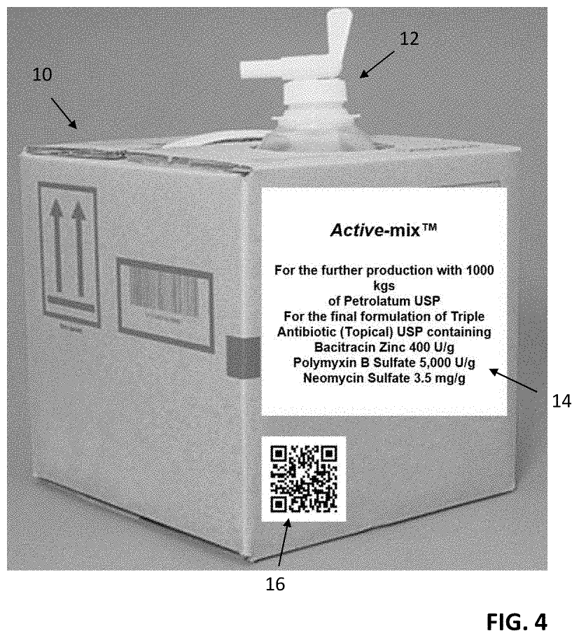

Referring to FIGS. 1-4, non-limiting examples of containers 10 for containing the highly concentrated formulation to be admixed with a vesseled excipient are shown. Container types may include, but are not limited to, foil pouches, ABL foil bag, rigid plastic containers, trays, bags, boxed bags, etc. Containers may be rigid or flexible and may withstand heating. Containers may configured for one-time use or may be re-sealable.

Foil pouches and bags, among others, are configured to withstand heating, while flexible containers provide the ability to further mix, handle and dispense the bag contents. Containers may optionally include handles to facilitate ergonomic handling for inverting the container. Containers may further include a discharge port 12 to facilitate transfer from the container when introducing the contents into a vessel or secondary excipient. The container may be packaged or contained within a nitrogen blanket or under compressed air, pressure or vacuum, among others, to increase the stability of the highly concentrated formulation.

Indicia 14 are provided on the container, such as printed directly thereon or applied to a label. The indicia 14 indicates one or more of the at least one API, the second inactive excipient paired for use with the highly concentrated formulation, the pharmaceutical formulation to be produced, the second API potency, and an amount of the second inactive excipient or the pharmaceutical formulation to be produced when the highly concentrated formulation is admixed with the second inactive excipient, among other information including, but not limited to, product name, brand name, expiration date, lot number, manufacturer, country of manufacture, etc. The container 10 may be color-coded for use with a matching color-coding of a vessel. The container 10 may further include a barcode, such as a quick response (QR) code as depicted. The container 10 may be labeled for use with a compatible vessel and/or second excipient volume, size, capacity or other indicative measurement.

In a first non-limiting example, the highly concentrated formulation may be packaged in a container and labeled indicated for use with a predetermined amount of excipient in a vessel, for example, a vessel containing approximately 1000 kgs volume of excipient paired for use with the highly concentrated formulation including approx. 3,500,000 mg of Neomycin Sulfate, 5,000,000,000 units of Polymyxin B Sulfate, and 400,000,000 units Bacitracin or Bacitracin Zinc.

In another non-limiting example, the highly concentrated formulation may be packaged in a container and labeled indicated for use with a vessel containing approximately 1000 kgs volume of excipient, the highly concentrated formulation including approximately. 3,000,000 to 5,000,000 mg of Neomycin Sulfate, 4,000,000,000 to 11,000,000,000 units of Polymyxin B Sulfate, and 400,000,000 to 700,000,000 units of Bacitracin or Bacitracin Zinc.

In some embodiments, the container may be labeled to indicate the name of the pharmaceutical formulation to be produced, the predetermined potency of the API(s) contained within the highly concentrated formulation and/or resulting potency after the combination of the highly concentrated formulation with the second excipient and/or the pharmaceutical formulation monograph and/or the volume of excipient to be combined with highly concentrated formulation.

The term “vessel” may refer to any mixing or holding tank used to at least one of mix, heat, store, transfer and homogenize the highly concentrated formulation and the paired excipient, and optionally any other raw material used in the manufacture of a pharmaceutical formulation such as a topical product. The term “excipient” may refer to any one or more raw material used for the further production of a pharmaceutical formulation such as a topical ointment, cream, ophthalmic or other formulation. The term “potency” may refer to a measurement of the concentration, strength or activity of a medication as the amount of API(s) within a predetermined unit measurement of the medication; however, this disclosure also recognizes the use of other generally related and exchangeable terminology for determining the same. The term “purity” may refer to a measurement of the pureness of a raw material such as an API and is most often used to determine the factor used to adjust for the amount of impurities within an API raw material; however, this disclosure also recognizes the use of other generally related and exchangeable terminology for determining the same.

In some embodiments, the highly concentrated formulation may include, in addition to at least one API and at least one excipient, at least one agent or additive to facilitate the introduction, admixing or homogenous distribution of the highly concentrated formulation into the vessel excipient.

In some embodiments, the highly concentrated formulation may include a corticosteroid such as hydrocortisone, and an excipient and/or viscosity agent such as cetyl palmitate. In one non-limiting example, a vessel containing about 950 to 1050 kgs volume of excipient is intended for use with the highly concentrated formulation containing about 8-12 kg of Hydrocortisone, or alternatively about 20-30 kg of Hydrocortisone. In another non-limiting example, the highly concentrated formulation may include hydrocortisone or other corticosteroid in a potency concentration level to achieve the activation of the complete contents of a specific volume of excipient in a vessel to achieve a final active potency of the API in a pharmaceutical formulation of about 1% or 2.5%, or between 0.8% and 5%.

In some embodiments, the highly concentrated formulation may include two or more APIs from the group including triamcinolone, neomycin, nystatin, gramicidin, or other combined anti-infective and/or steroid formulation compound. In a non-limiting formulation example, the highly concentrated formulation may include a plurality of APIs from the group including triamcinolone, neomycin, nystatin, and gramicidin formulated to achieve a final formulation within the excipient in a vessel containing 1 mg triamcinolone acetonide, 2.5 mg neomycin base (as sulfate), 0.25 mg gramicidin, and 100,000 nystatin units per gram, among others.

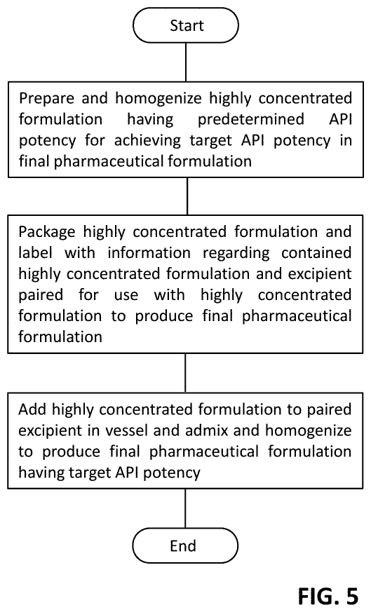

With reference to FIG. 5, the inventive concepts disclosed herein are further directed to identifying and compounding a first inactive excipient, and optional viscosity agent, with a predetermined amount of at least one API to produce a highly concentrated formulation having a predetermined API potency, mesh size and ratio of API/excipient compatible for the introduction, admixing and dissolution within a second inactive excipient, wherein an amount of the second inactive excipient is greater than an amount of the first inactive excipient such an API potency of the highly concentrated formulation is greater than an API potency of the highly concentrated formulation and second inactive excipient admixed together.

The highly concentrated formulation is paired to the second inactive excipient and is provided in an amount for admixing with a predetermined amount of the second inactive excipient to produce a large batch of pharmaceutical formulation having a predetermined API potency according to a health agency pharmacopeia or monograph USP, EP, JP, IN, among others, or other intended specification.

Preparing a highly concentrated formulation may include the steps of providing a first API, the first API purity tested through various industry known analytical testing procedures, and calculating an amount of the first API (based on the purity) needed to achieve a predetermined first API purity in a final (e.g., large batch) pharmaceutical formulation, optionally providing a second API, the second API purity tested as above, and calculating an amount of the second API (based on the purity) needed to achieve a predetermined second API purity in the final pharmaceutical formulation, optionally providing a third and additional APIs as above, admixing the calculated amount of first API and optional additional APIs with a predetermined amount of a compatible first inactive excipient and optional additive(s), homogenizing the admixed highly concentrated formulation, and packaging and labeling the homogenized highly concentrated formulation for use with a predetermined paired excipient of a predetermined amount. Preparation may further optionally include conducting stability studies according to ICH regulatory requirements among other stability protocols on the first composition and the API(s) contained therein within various temperature and humidity levels and within various packaging conditions.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed in:

1. A packaged formulation for producing a batch of a pharmaceutical formulation, comprising:
a container;
a highly concentrated formulation contained in the container, the highly concentrated formulation comprising at least one active pharmaceutical ingredient (API) admixed with a first inactive excipient, the highly concentrated formulation having a first API potency to produce a pharmaceutical formulation having a second API potency less than the first API potency and in compliance with a predetermined regulatory pharmacopeia or formulary monograph for the produced pharmaceutical formulation when the highly-concentrated formulation is admixed with a second inactive excipient; and
indicia on the container indicating the at least one API, the second inactive excipient paired for use with the highly concentrated formulation, the pharmaceutical formulation to be produced, the second API potency, and an amount of the second inactive excipient or the pharmaceutical formulation to be produced when the highly concentrated formulation is admixed with the second inactive excipient.

2. The packaged formulation according to claim 1, wherein the at least one API is an antimicrobial.

3. The packaged formulation according to claim 1, wherein the at least one API is a steroid.

4. The packaged formulation according to claim 1, wherein the at least one API is biologically active.

5. The packaged formulation according to claim 1, wherein the first API potency is at least 30 times greater than the second API potency.

6. The packaged formulation according to claim 1, wherein the first API potency is at least 50 times greater than the second API potency.

7. The packaged formulation according to claim 1, wherein the first API potency is at least 100 times greater than the second API potency.

8. The packaged formulation according to claim 1, wherein the amount of the second inactive excipient is at least 30 times greater than the amount of the first inactive excipient.

9. The packaged formulation according to claim 1, wherein the amount of the second inactive excipient is at least 50 times greater than the amount of the first inactive excipient.

10. The packaged formulation according to claim 1, wherein the amount of the second inactive excipient is at least 100 times greater than the amount of the first inactive excipient.

11. The packaged formulation according to claim 1, wherein the first inactive excipient and the second inactive excipient are the same, and wherein at least one of the first inactive excipient and the second inactive excipient is in a form of a liquid, a gel, a semi-solid, or a solid at room temperature.

12. The packaged formulation according to claim 1, wherein the pharmaceutical formulation is a topical or ophthalmic formulation.

13. The packaged formulation according to claim 1, wherein the indicia is a barcode or a barcode is provided on the container in addition to the indicia.

14. The packaged formulation according to claim 1, wherein the highly concentrated formulation comprises two or more APIs.

15. A method for producing a pharmaceutical formulation, comprising:
providing a packaged formulation comprising:
a container;
a highly concentrated formulation contained in the container, the highly concentrated formulation comprising at least one active pharmaceutical ingredient (API) admixed with a first excipient, the highly-concentrated formulation having a first API potency to produce a pharmaceutical formulation having a second API potency less than the first API potency and in compliance with a predetermined regulatory pharmacopeia or formulary monograph for the produced pharmaceutical formulation when the highly-concentrated formulation is admixed with a second inactive excipient; and
indicia on the container indicating the at least one API, the second inactive excipient paired for use with the highly concentrated formulation, the pharmaceutical formulation to be produced, the second API potency, and an amount of the second inactive excipient or the pharmaceutical formulation to be produced when the highly concentrated formulation is admixed with the second inactive excipient; and
admixing the highly concentrated formulation with the second excipient contained in a vessel, wherein an amount of the second excipient is greater than an amount of the first excipient.

16. The method according to claim 15, wherein the at least one API is an antimicrobial, a steroid, or is biologically active.

17. The method according to claim 15, wherein the amount of the second inactive excipient is at least 75 times greater than the amount of the first inactive excipient, and wherein the first API potency is at least 50 times greater than the second API potency.

18. The method according to claim 15, wherein the first inactive excipient and the second inactive excipient are the same, and wherein at least one of the first inactive excipient and the second inactive excipient is a liquid, a gel, a semi-solid, or a solid at room temperature.

19. The method according to claim 15, wherein the highly concentrated formulation comprises two or more APIs.

20. The method according to claim 15, wherein the pharmaceutical formulation is a topical or ophthalmic formulation.
